(19) United States
(12) Patent Application Publication
(10) Pub. No.: US 2026/0060548 A1
(43) Pub. Date: Mar. 5, 2026

(54) Housing for a Sensor and Sensor

(71) Applicant: Eagle Actuator Components GmbH & Co. KG, Heppenheim (DE)

(72) Inventors: Ralf Heinrich, Schwegenheim (DE); Daniel Koch, Edingen-Neckarhausen (DE); Siegfried Driemer, Fürth (DE); Christiane Spilger, Fürth (DE)

(73) Assignee: Eagle Actuator Components GmbH & Co. KG, Heppenheim (DE)

(21) Appl. No.: 19/276,863

(22) Filed: Jul. 22, 2025

(30) Foreign Application Priority Data
Aug. 30, 2024 (DE) 102024124894.7

(51) Int. Cl. A61B 5/00; A61B 5/20

(52) U.S. Cl. CPC A61B 5/0071; A61B 5/201; A61B 2562/166

(57) ABSTRACT

A housing for a sensor, comprising an upper housing part and a lower housing part which, when assembled, form a receiving space suitable for receiving a circuit board with at least one LED which emits light through the lower housing part. A plurality of passages are formed in the lower housing part, each of which is separated from the others by at least one partition wall, and at least one partition wall is connected to a receptacle or receiving tray formed in or on the lower housing part for an optical filter. A structure for guiding light is formed or arranged in or on the lower housing part and oriented toward the optical filter. The housing enables a sensor to receive light signals as reliably and clearly as possible, independently of the angles of incidence of the light returning to the sensor from a human or animal body.

### FIELD OF THE DISCLOSURE

The present disclosure relates to a sensor and a housing for a sensor.

### BACKGROUND OF THE DISCLOSURE

Sensors may be used to examine the function of human or animal organs by injecting a marker into them. For example, a marker may include a substance that can be excited to fluoresce when exposed to excitation light. The decrease in fluorescence over time can be seen as an indicator of how well an organ, especially a kidney, breaks down or excretes the marker or substance. The sensor can be worn close to the body to detect fluorescence.

### SUMMARY OF THE DISCLOSURE

The present disclosure is directed to systems and methods for sensors and housings for sensors. In some implementations, a housing may comprise an upper housing part and a lower housing part which, when assembled, form a receiving space suitable for receiving a circuit board with at least one light emitting diode (LED) which emits light through the lower housing part. In some implementations, a plurality of passages are formed in the lower housing part, each of which is separated from the others by at least one partition wall, and at least one partition wall is connected to a receptacle or receiving tray formed in or on the lower housing part for an optical filter. In some implementations, a sensor may receive light signals to be detected as reliably and clearly as possible, independently of the angles of incidence of the light returning to the sensor from a human or animal body. In many implementations, a structure for guiding light is formed or arranged in or on the lower housing part and oriented toward the optical filter.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 shows an exploded view of a sensor comprising, in the drawing plane from top to bottom, an upper housing part, a circuit board, a lower housing part, and an optical filter, wherein a structure, namely a perforated grid, is formed in the lower housing part;

FIG. 2 shows a schematic cross-sectional view, not to scale in terms of structure, of the sensor according to FIG. 1 in assembled state, showing that only light reflected from the patient's skin falls on the photodiode, which has previously fallen on the optical filter at an angle of incidence of less than 17°, because the schematically-represented structure geometrically forces this;

FIG. 3 shows the housing according to FIG. 2, which accommodates the circuit board inside and carries the filter, thus forming a sensor, wherein the sensor is adhered to the skin of a patient with the filter facing forward, and wherein light is schematically represented, which is emitted by the LEDs onto the skin of the patient and reflected from there, then passing through the schematically represented structure to the photodiode; and

FIG. 4 shows a schematic representation of the transmission behavior of an optical filter as a function of the wavelength of the light incident on it.

### DETAILED DESCRIPTION

DE 10 2023 104 302 A1 describes an implementation of a housing and a sensor, which can be used to examine the function of human or animal organs by injecting a marker into them.

In some such implementations, the marker contains a substance that can be excited to fluoresce when exposed to excitation light. The decrease in fluorescence over time can be seen as an indicator of how well an organ, especially a kidney, breaks down or excretes the marker or substance. The sensor can be worn close to the body to detect fluorescence.

EP 0 966 306 B1 describes an implementation of a method for measuring physiological functions, including injecting a patient with fluorophores or chromophores that fluoresce when excited by light and are excreted by kidney cells over time. This allows kidney function to be examined.

EP 2 317 911 B1 describes an implementation of a sensor patch comprising an excitation light source and a detector. EP 2 895 073 B1 describes an implementation of an adhesive functional strip with a sensor head with LED light and a photodiode.

The housing of the sensor in accordance with DE 10 2023 104 302 A1 mentioned above is subject to special requirements. The housing must conduct excitation light from LEDs to the human or animal body and reliably feed fluorescent light, which is reflected back to the sensor in response to the excitation light, to a photodiode.

As little extraneous light as possible, i.e., light superimposed on the fluorescence light, should fall on the photodiode. Against this background, DE 10 2023 104 302 A1 already discloses an optical filter that rests against a partition wall with as little gap as possible and allows excitation light from the LEDs to pass through in the direction of emission, but is intended to be largely opaque to excitation light falling back toward the photodiode.

Of course, the optical filter should allow fluorescent light reflected from a marker in the human or animal body to pass through so that the photodiode can detect it.

Glass filters or thin-film filters are known from the prior art, which block blue light from LEDs and allow green light, i.e., fluorescent light, to pass through.

However, a transmission curve for such a thin-film filter only shows the optical conditions when light strikes the filter at a right angle.

In fact, the transmission behavior of a filter may be angle-dependent with respect to the angle of incidence of the light beam. The frequency band to be filtered by the filter can be influenced by the angle of incidence of the incident light. The frequency band can shift toward shorter wavelengths, allowing light with shorter wavelengths to pass through the filter more easily and be blocked less than desired when the light strikes the filter at a greater angle of incidence. The following approximation applies to the shift by a wavelength interval:

$$Δλ \approx k \cdot {sin}^{2} α$$

In this formula, Δλ is the wavelength interval by which the shift occurs, and a is the angle of incidence.

During a measurement, blue excitation light reflected from a patient's skin may therefore strike the filter at an angle of incidence that deviates from 90°. As a result, the returning blue light is blocked less by the filter than it would be at a 90° angle, and overlaps the fluorescent light. The photodiode can therefore also receive signals from light that it is not actually supposed to detect.

Implementations of the systems and methods discussed herein address the aforementioned and other problems through providing a sensor that receives signals of the light to be detected as reliably and clearly as possible, regardless of the angles of incidence of the light reflected back to the sensor from a human or animal body.

According to some implementations of these systems and methods, certain light may be transmitted through a structure to a photodiode in order to avoid signal distortion. In some implementations of these systems and methods, an optical filter may be combined with a structure that allows only light rays that have previously fallen onto the optical filter at a small angle of incidence to pass through to the photodiode, while blocking, deflecting, or suppressing other light rays as much as possible. The light rays that hit the optical filter mostly vertically or at a really small angle may be sorted by the optical filter based on what they are used for, and may be let through or passed through the structure. Such implementations allow defined light of a specific frequency range to pass through, and blocks light of another frequency range or ranges. In some implementations of these systems and methods, the structure may be assigned to the housing in order to achieve a simple and compact design of the housing and to keep the number of components to a minimum.

In some implementations, the housing or structure may be designed as a perforated grid, with one or more holes forming a light channel or leading into a light channel. The structure thus uses mechanical means to create a geometric boundary for light that could potentially fall back onto the photodiode, so that only light that has previously fallen onto the optical filter at a small angle of incidence can reach the photodiode.

In some implementations, the housing or structure may be designed as a perforated grid, with at least two holes separated by a web whose width is in the range of 0.01 to 1 mm. This allows a large number of light channels to be created and the signal of the returning light to be detected to be increased.

In some implementations, the housing or structure may be designed as a perforated grid, with at least one hole having a diameter or width in the range of 0.01 to 0.8 mm. The perforated grid thus creates a geometric boundary for returning light, so that only returning light that has previously fallen on the optical filter at an angle of incidence <17° can reach the photodiode. Such light was also filtered correctly by the optical filter in accordance with the specific application.

In some implementations, the housing or structure may be designed as a perforated grid, with at least one hole acting as a light channel having a length in the range of 0.5 to 2 mm. This allows light that has fallen on the optical filter at a large angle of incidence to run into the wall of the light channel and not reach the photodiode. Light can be absorbed preferentially in the wall of the light channel.

In some implementations, the housing or structure may be arranged and formed between two non-transparent partition walls, with each partition wall separating two passages in the lower housing part from each other. This prevents excitation light from an LED from falling directly onto the photodiode and also ensures that excitation light reflected from the patient's skin, as well as fluorescent light, must first pass through the structure before it can reach the photodiode.

In some implementations, an upper housing part and/or a lower housing part of the structure may be made of a plastic material which is free of glass, in particular free of glass fibers. This prevents light from being conducted within housing parts and distorting signals. In some implementations, PA 6 may be used as the plastic material.

In some implementations, a sensor may comprise a housing of the type described above and a circuit board, wherein the circuit board carries two LEDs for emitting excitation light and a photodiode for detecting fluorescent light, wherein the LEDs each protrude into a first and a third passage, wherein the photodiode protrudes into a second passage which opens into the structure, wherein the passages for the LEDs are each separated from the second passage for the photodiode by an opaque partition wall, and wherein an optical filter is accommodated in a receiving tray.

With such a sensor, measurements can be taken close to the body for an hour or longer. The sensor can be worn close to the body and the patient being examined can move around largely unhindered while wearing the sensor. The recorded measurement data can be read from the sensor after it has been removed from the patient.

In some implementations, a microprocessor or microcontroller that can evaluate the data detected by the photodiode may also be provided on the circuit board. In some implementations, the microprocessor can be read by inserting a plug into a plug-in connection in the sensor. The plug-in connection can also be used to connect a battery to the circuit board, which supplies power to the LEDs and electronics during measurement. In other implementations, the connection may be wireless or by any other suitable means.

In some implementations, the optical fiber may comprise a glass plate carrying an optical thin-film filter. This makes it possible to use a stable and sufficiently thick glass plate that is only partially coated with a thin layer.

In some implementations, only one third of the surface of the glass plate that is in contact with the structure should be coated. Only the coated part of the optical filter is in close contact with the structure.

In some implementations, the glass plate may be 1 mm thick. The optical filter may be designed as a long-pass filter. The optical filter allows longer wavelengths to pass through and blocks shorter wavelengths. This blocks the blue light from the LEDs and allows green light to pass through as fluorescent light to the photodiode.

In some implementations, an arrangement or device may comprise a sensor of the type described here and a light-tight adhesive film which is arranged on the lower housing part and has a recess through which both excitation light and reflected light can pass. This adhesive film allows the sensor to be worn close to the body while preventing light from being lost through lateral gaps during measurement.

The sensor is preferably used as an organ function sensor, in particular as a kidney function sensor.

In the description that follows, reference is made to the following elements as shown in the accompanying drawings:
1 Housing
2 Sensor
3 Upper housing part of 1
3a Lid of 3
4 Lower housing part of 1
5 Receiving space in 1
6 Circuit board
7 Structure, perforated grid
8a Blue light spectrum of an LED for excitation
8b Green light from marker
8c Transmission behavior of 12, 13a, 13b
9 Support base
9a Receiving tray in 4 for 6
9b Additional receiving tray in 4 for 12
10a First passage in 9
10b Second passage in 9
10c Third passage in 9
11a First partition wall
11b Second partition wall
12 Optical filters
13a Glass plate
13b Optical thin-film filter
15a First LED
15b Second LED
16 Photodiode
17 Microprocessor or microcontroller
18 Plug-in connection
19 Light-tight adhesive film
19a Skin
20 Web in 7
20a Width of 20
21 Hole in 7
22 Recess in 19

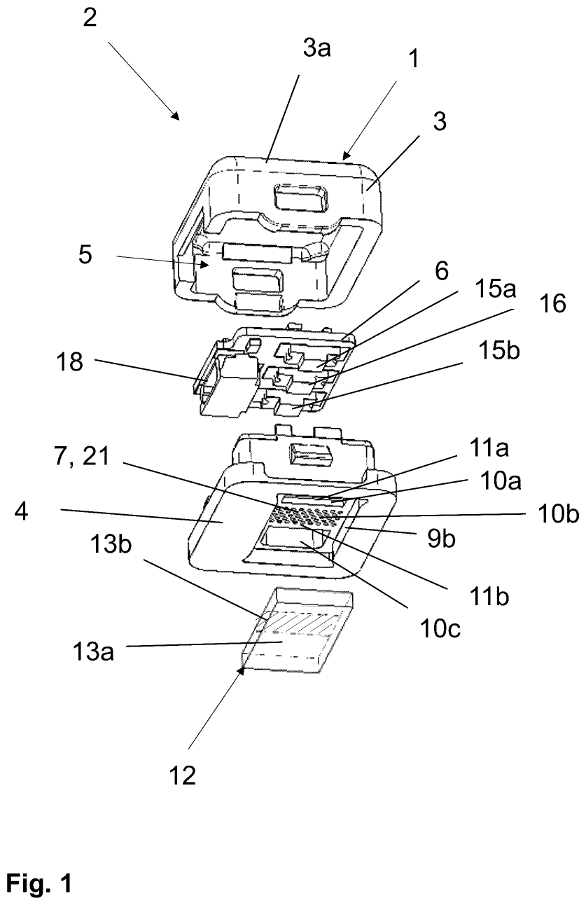

FIG. 1 shows an exploded view of a sensor 2 with a housing 1. The housing 1 for the sensor 2 comprises an upper housing part 3 and a lower housing part 4, which, when assembled, form a receiving space 5 that is suitable for receiving a circuit board 6 with at least one LED 15a, 15b that emits light through the lower housing part 4.

A plurality of passages 10a, 10b, 10c are formed in the lower housing part 4, each of which is separated from the others by at least one opaque partition wall 11a, 11b. At least one partition wall 11a, 11b is connected to a receiving tray 9b for an optical filter 12, which is formed in the lower housing part 4, in the direction of radiation of the LEDs 15a, 15b.

A structure 7 for guiding light is formed in the lower housing part 4, which can be turned or oriented toward the optical filter 12. Specifically, a structure 7 for guiding light is formed in the lower housing part 4, to which the optical filter 12 can be applied without gaps.

Structure 7 is made of one piece and is made of the same material as the lower housing part 4. Structure 7 is designed as a sieve-like perforated grid with a large number of holes 21.

The housing 1 consists of only two parts, namely the upper housing part 3 and the lower housing part 4.

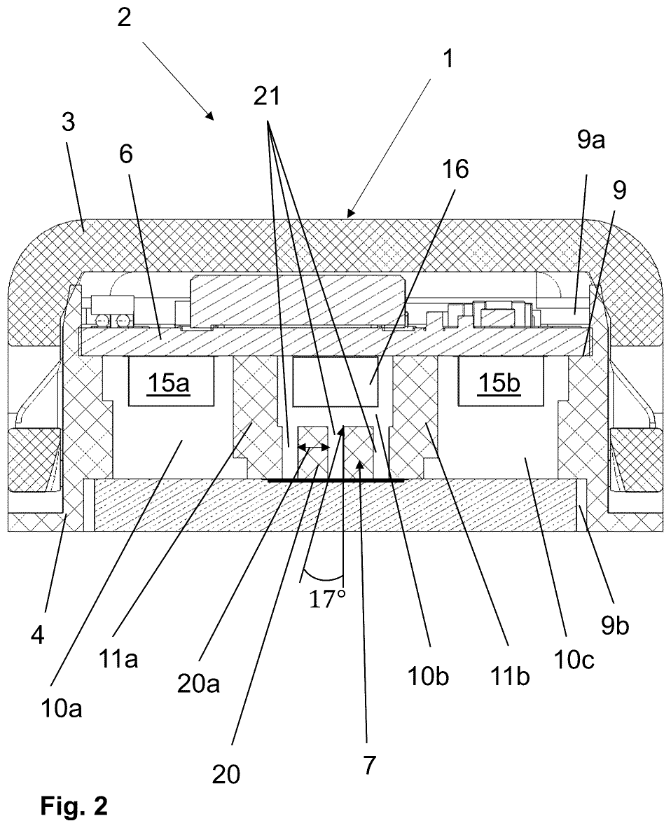

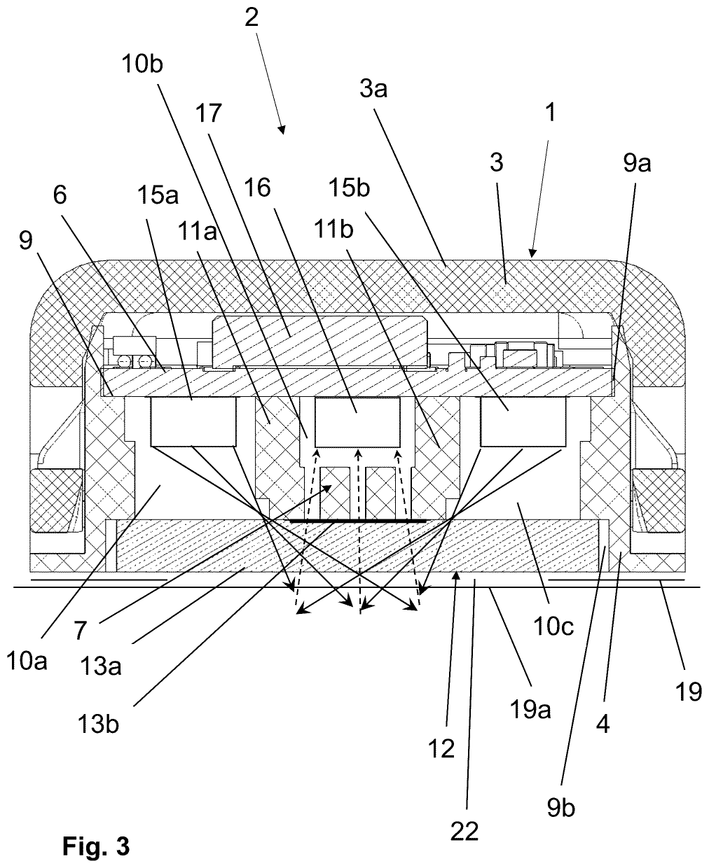

FIG. 2 shows schematically, by means of a structure 7 that is not to scale, particularly in relation to FIG. 1, that the structure 7 is designed as a perforated grid, wherein each hole 21 forms a light channel. Structure 7 is designed as a perforated grid, wherein at least two holes 21 are separated from each other by a web 20, the width 20a of which is at least 0.3 mm. The structure 7 is designed as a perforated grid, wherein at least one hole 21 has a diameter or width of 0.3 mm. The structure 7 is designed as a perforated grid, wherein at least one hole 21 has a length of 1 mm as a light channel. FIG. 2 and FIG. 3 show only three holes 21 in schematic enlargement.

FIG. 2 shows specifically that only light that has previously fallen onto the optical filter 12 at a small angle of incidence is transmitted through the structure 7 to fall onto the photodiode 16.

FIGS. 1 through 3 show that structure 7 is arranged and formed between two opaque partition walls 11a, 11b, each partition wall 11a, 11b separating two passages 10a, 10b, 10c in the lower housing part 4 from each other.

The upper housing part 3 and the lower housing part 4 are made of PA6 plastic, which is glass-free and, in particular, does not contain any glass fibers or glass particles. Structure 7 is also made from this type of plastic.

FIGS. 2 and 3 show a sensor 2 comprising a housing 1 and a circuit board 6, wherein the circuit board 6 carries two LEDs 15a, 15b for emitting excitation light and a photodiode 16 for detecting fluorescent light, wherein the LEDs 15a, 15b are each inserted into a first and a third passage 10a, 10c, the photodiode 16 protruding into a second passage 10b which opens into the structure 7 and merges into it, the passages 10a, 10c for the LEDs 15a, 15b each being separated from the second passage 10b for the photodiode 16 by an opaque partition wall 11a, 11b, and wherein an optical filter 12 is accommodated in the receiving tray 9b formed in the lower housing part 4.

The optical filter 12 is aligned with the outer walls of the lower housing part 4 and can thus be placed flat and evenly on the skin 19a of a patient without creating lateral gaps. The optical filter 12 fits snugly against the structure 7, which is made of an opaque plastic.

FIGS. 1 through 3 show that the optical filter 12 comprises a glass plate 13a which supports an optical thin-film filter 13b. Only the middle third of the surface of the glass plate 13a facing the LEDs 15a, 15b and the photodiode 16 is coated with the thin-film filter 13b. Specifically, only the area of the surface that is in contact with structure 7 without any gaps is coated.

FIG. 3 shows an arrangement comprising the sensor 2 according to FIG. 2 and a light-tight adhesive film 19 which is arranged on the lower housing part 4 and has a recess 22 through which both excitation light and reflected light can pass.

FIGS. 2 and 3 show specifically that a receiving tray 9a with a support base 9 is formed for the circuit board 6 in the lower housing part 4. The support base 9 is connected to the further receiving tray 9b for the optical filter 12, wherein the further receiving tray 9b for the optical filter 12 is located opposite the receiving tray 9a for the circuit board 6 in the vertical direction. The upper housing part 3 has a closed lid 3a so that the electronics of the circuit board 6 are protected.

The circuit board 6 carries two LEDs 15a, 15b for emitting blue excitation light and at least one photodiode 16 for detecting green fluorescent light, wherein the LED 15a protrudes into a first passage 10a in the support base 9, wherein the photodiode 16 protrudes into a second passage 10b in the support base 9, wherein the second LED 15b protrudes into a third passage 10c in the support base 9, wherein the passages 10a, 10b are separated from each other by a first opaque partition wall 11a and wherein the passages 10b, 10c are separated from each other at the sides by a second opaque partition wall 11b.

The partition walls 11a, 11b are connected at their upper ends to the circuit board 6 and form part of the support base 9 or are aligned with it and with each other. The partition walls 11a, 11b lie with their lower ends against the optical filter 12 without any gaps. Structure 7 also fits tightly against optical filter 12 without any gaps.

A microprocessor 17 is arranged on circuit board 6, which can evaluate the data detected by photodiode 16. The microprocessor 17 can be read out by inserting a plug into a plug-in connection 18 in the sensor 2.

The plug-in connection 18 can also be used to connect a battery to the circuit board 6, which supplies power to the LEDs 15a, 15b and the rest of the electronics during measurement.

Towards the bottom, i.e., in the direction of the excitation light exit, housing 1 is open for light to pass through at least through passages 10a through 10c, but is mechanically closed by optical filter 12 so that the interior of sensor 2 cannot be accessed from below.

Only through the opening for the plug-in connection 18 can a battery plug be connected electrically and mechanically to the circuit board 6. The plug-in connection 18 is also used to read data stored in the microprocessor 17 or in a memory. The plug-in connection 18 is shown in FIG. 1.

The width and/or length of the housing 1 should preferably be less than 1 cm. The height of the housing 1 should preferably be less than 1 cm.

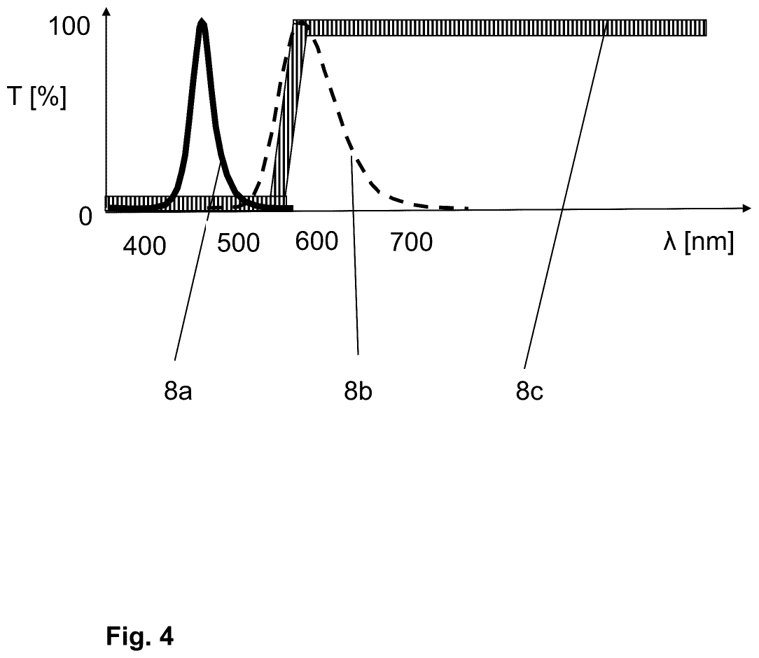

FIG. 4 schematically shows that blue light in the wavelength range 400 to 500 nm, which is emitted by LEDs 15a, 15b as excitation light, is almost completely blocked by a long-pass filter, so that the transmission T with respect to this light is almost 0%. FIG. 4 further shows schematically that green light from the wavelength range 500 to 700 nm, which is reflected back as fluorescent light from a marker in the patient onto the photodiode 16, is predominantly transmitted by a long-pass filter, so that the transmission T with respect to this light is almost 100%, at least above 600 nm.

However, the transmission behavior shown assumes that both the blue and green light fall perpendicularly on the long-pass filter. As soon as blue light hits the long-pass filter at an angle of incidence other than 0°, it is transmitted more strongly by the long-pass filter because its transmittance increases in relation to shorter-wave light.

However, the structure 7 described here ensures that shorter-wave light, which should actually have been filtered out by the long-pass filter, does not reach the photodiode 7 as far as possible. The structure 7 ensures that only light that has actually fallen on the optical filter 12 at a small angle of incidence and has been filtered according to the specific application reaches the photodiode 16.

What is claimed:

1. A housing for a sensor, comprising:
an upper housing part and a lower housing part which, when assembled, form a receiving space suitable for receiving a circuit board with at least one light emitting diode (LED) which emits light through the lower housing part,
wherein a plurality of passages are formed in the lower housing part, which are each separated from one another by at least one partition wall, and
wherein at least one partition wall of the lower housing part forms a receptacle or receiving tray for an optical filter, and
wherein a structure for guiding light is formed or arranged in or on the lower housing part and oriented toward the optical filter.

2. The housing of claim 1, wherein the structure comprises a perforated grid comprising a plurality of holes, wherein each hole forms a light channel or leads into a light channel.

3. The housing of claim 1, wherein the structure comprises a perforated grid comprising a plurality of holes, wherein at least two holes are separated from each other by a web whose width is in the range of 0.01 to 1 mm.

4. The housing of claim 1, wherein the structure comprises a perforated grid comprising a plurality of holes, wherein at least one hole has a diameter or width which is in the range of 0.01 to 0.8 mm.

5. The housing of claim 1, wherein the structure comprises a perforated grid, wherein at least one hole has a length as a light channel which is in the range of 0.5 to 2 mm.

6. The housing of claim 1, wherein the structure is arranged and formed between two non-transparent partition walls, each partition wall separating two passages in the lower housing part from each other.

7. The housing of claim 1, wherein the upper housing part and/or the lower housing part is or are made of a plastic which does not contain any glass fibers.

8. A sensor, comprising:
a circuit board comprising two light emitting diodes (LEDs) for emitting excitation light and a photodiode for detecting fluorescent light;
an optical filter; and
a housing comprising an upper housing part and a lower housing part which, when assembled, form a receiving space suitable for receiving the circuit board,
wherein a plurality of passages are formed in the lower housing part, each separated from one another by at least one partition wall, and
wherein at least one partition wall of the lower housing part forms a receptacle or receiving tray for the optical filter, and
wherein a structure for guiding light is formed or arranged in or on the lower housing part and oriented toward the optical filter;
wherein the LEDs each project into a first and a third passage of the plurality of passages, the photodiode projects into a second passage of the plurality of passages which opens into the structure, and the passages for the LEDs are each separated from the second passage for the photodiode by an opaque partition wall.

9. The sensor of claim 8, wherein the optical filter comprises a glass plate which carries an optical thin-film filter.

10. The sensor of claim 8, further comprising a light-tight adhesive film which is arranged on the lower housing part and has a recess through which both excitation light and reflected light can pass.

11. The sensor of claim 8, wherein the structure comprises a perforated grid comprising a plurality of holes, wherein each hole forms a light channel or leads into a light channel.

12. The sensor of claim 8, wherein the structure comprises a perforated grid comprising a plurality of holes, wherein at least two holes are separated from each other by a web whose width is in the range of 0.01 to 1 mm.

13. The sensor of claim 8, wherein the structure comprises a perforated grid comprising a plurality of holes, wherein at least one hole has a diameter or width which is in the range of 0.01 to 0.8 mm.

14. The sensor of claim 8, wherein the structure comprises a perforated grid, wherein at least one hole has a length as a light channel which is in the range of 0.5 to 2 mm.

15. The sensor of claim 8, wherein the structure is arranged and formed between two non-transparent partition walls, each partition wall separating two passages in the lower housing part from each other.

16. The sensor of claim 8, wherein the upper housing part and/or the lower housing part is or are made of a plastic which does not contain any glass fibers.
